(19) United States
(12) Patent Application Publication
(10) Pub. No.: US 2017/0296067 A1
(43) Pub. Date: Oct. 19, 2017

(54) INTELLIGENT WEARABLE DEVICE AND DETECTION METHOD OF BIOMETRIC INFORMATION

(71) Applicant: BOE TECHNOLOGY GROUP CO., LTD., Beijing (CN)

(72) Inventor: Jiuxia YANG, Beijing (CN)

(73) Assignee: BOE TECHNOLOGY GROUP CO., LTD., Beijing (CN)

(21) Appl. No.: 15/324,078

(22) PCT Filed: Jul. 26, 2016
(86) PCT No.: PCT/CN2016/091727

§ 371 Date: Jan. 5, 2017

(30) Foreign Application Priority Data
Nov. 24, 2015 (CN) 201510822638.8

(51) Int. Cl. A61B 5/0205; A61B 5/00; A61B 5/117; G06Q 20/40; A61B 5/1455; A61B 5/024

(52) U.S. Cl. CPC A61B 5/0205; G06Q 20/4014; A61B 5/6826; A61B 5/0022; A61B 5/117; A61B 5/02438; A61B 5/14552

(57) ABSTRACT

An intelligent wearable device and a detection method of biometric information of the intelligent wearable device. The intelligent wearable device includes: a wearable device body; and a light source, a first photoelectric detection module, a second photoelectric detection module and a processing module disposed on the wearable device body. The light source is configured to emit light towards a human-body wearing part; the light includes transmitted light that passes through the human-body wearing part and reflected light reflected by the human-body wearing part; the first photoelectric detection module is configured to detect the transmitted light; the second photoelectric detection module is configured to detect the reflected light; and the processing module is configured to determine a first type of biometric information of a human body according to the transmitted light and determine a second type of biometric information of the human body according to the reflected light.

### TECHNICAL FIELD

Embodiments of the present disclosure relate to an intelligent wearable device and a detection method of biometric information used in the intelligent wearable device.

### BACKGROUND

With the development of science and technology, more and more intelligent wearable products (e.g., intelligent wristlets and rings) enter people's lives. Taking an intelligent ring as an example, a user can record real-time data such as exercise, sleep and diet in daily life and synchronize the data on terminal devices such as mobile phones and tablet computers, so that an objective of achieving healthy life based on the data can be achieved.

Currently, most intelligent wearable devices are simple. Even if there are multiple available functions, functional modules corresponding to the functions are mutually independent and occupy a large space; the integration of the functions within limited space is low; and realizable functions are limited.

### SUMMARY

Embodiments of the present disclosure provide an intelligent wearable device, comprising: a wearable device body; and a light source, a first photoelectric detection module, a second photoelectric detection module and a processing module disposed on the wearable device body, where:

the light source is configured to emit light towards a human-body wearing part; the light includes transmitted light that passes through the human-body wearing part and reflected light reflected by the human-body wearing part;

the first photoelectric detection module is configured to detect the transmitted light;

the second photoelectric detection module is configured to detect the reflected light; and

the processing module is connected with the first photoelectric detection module and the second photoelectric detection module, and the processing module is configured to determine a first type of biometric information of a human body according to the transmitted light detected by the first photoelectric detection module and determine a second type of biometric information of the human body according to the reflected light detected by the second photoelectric detection module.

Embodiments of the present disclosure further provide a detection method of biometric information for an intelligent wearable device, comprising:

detecting transmitted light which is emitted towards a human-body wearing part and passes through the human-body wearing part, and detecting reflected light which is reflected by the human-body wearing part;

determining a first type of biometric information according to the detected transmitted light; and

determining a second type of biometric information according to the detected reflected light.

### BRIEF DESCRIPTION OF THE DRAWINGS

In order to illustrate the technical solutions in the embodiments of the present disclosure or the existing arts more clearly, the drawings need to be used in the description of the embodiments or the existing arts will be briefly described in the following; it is obvious that the drawings described below are only related to some embodiments of the present disclosure, for one ordinary skilled person in the art, other drawings can be obtained according to these drawings.

FIG. 1 is a schematic structural view of an intelligent wearable device provided by one embodiment of the present disclosure;

FIG. 2 is a schematic structural view of a processing module provided by one embodiment of the present disclosure;

FIG. 3 is a schematic structural view of a heart rate simulation calculator provided by one embodiment of the present disclosure;

FIG. 4 is a schematic diagram of a heart rate simulation curve provided by one embodiment of the present disclosure;

FIG. 5 is a flowchart of a detection method of biometric information provided by one embodiment of the present disclosure; and

FIG. 6 is a flowchart of a detection method of biometric information provided by another embodiment of the present disclosure.

### DETAILED DESCRIPTION

Embodiments of the present disclosure provide an intelligent wearable device and a detection method of biometric information, which can simultaneously detect multiple types of biometric information and hence improve the integration of the intelligent wearable device and simplify the structure of the intelligent wearable device.

In order to illustrate the objectives, the technical solutions and advantages of the present disclosure more clearly, embodiments of the present disclosure will be described below in more details. It is obvious that the described embodiments are just a part but not all of the embodiments of the present disclosure. Based on the embodiments of the disclosure, other embodiments obtained from the present disclosure by those skilled in the art without making additional inventive work should be within the scope of the present disclosure.

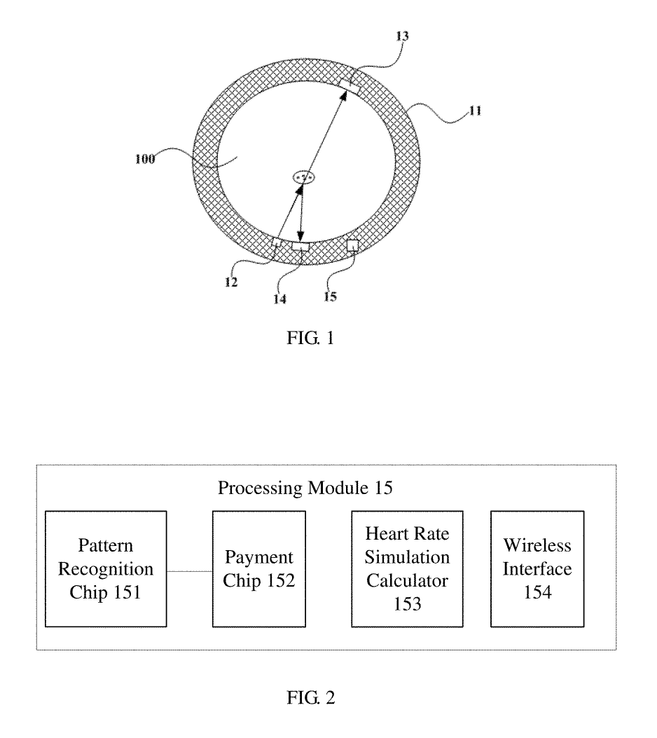

As illustrated in FIG. 1, an intelligent wearable device provided by an embodiment of the present disclosure comprises a wearable device body 11, and a light source 12, a first photoelectric detection module 13, a second photoelectric detection module 14 and a processing module 15 that are disposed on the wearable device body 11, where: the light source 12 is configured to emit light towards a human-body wearing part 100; the light includes transmitted light that passes through the human-body wearing part 100 and reflected light reflected by the human-body wearing part 100; the first photoelectric detection module 13 is configured to detect the transmitted light; the second photoelectric detection module 14 is configured to detect the reflected light; and the processing module 15 is connected with the first photoelectric detection module 13 and the second photoelectric detection module 14 and configured to determine a first type of biometric information of a human body according to the transmitted light detected by the first photoelectric detection module 13 and determine a second type of biometric information of the human body according to the reflected light detected by the second photoelectric detection module 14. For instance, the processing module 15 may be communicated with the first photoelectric detection module 13 and the second photoelectric module 14 by a wired connection and/or a wireless connection.

The biometric information of the human body may be detected by adoption of the technical solution of the embodiment, so that the health condition of the human body can be obtained. Moreover, the intelligent wearable device can simultaneously detect multiple types of biometric information. Since a same light source is adopted to provide detection light for the first photoelectric detection module and the second photoelectric detection module, the structure of the intelligent wearable device is simple and the cost of the intelligent wearable device is low.

In the embodiments of the present disclosure, a specific type of the intelligent wearable device is not limited, for instance, the intelligent wearable device may be an intelligent ring, an intelligent watch, an intelligent wristlet, etc. In one embodiment of the present disclosure, the intelligent wearable device is an intelligent wristlet and may be worn around a wrist of a human body. For instance, the intelligent wearable body is a wristlet body; the light source, the first photoelectric detection module and the second photoelectric detection module are disposed on an inner side of the wristlet body; the light source is arranged towards the first photoelectric detection module; and the second photoelectric detection module is disposed on a reflection path of light reflected by the wrist.

In the embodiments of the present disclosure, the biometric information determined by the transmitted light that passes through the human body is the first type of biometric information. For instance, the first type of biometric information may include venous information.

In the embodiments of the present disclosure, the biometric information determined by the reflected light that is reflected by the human body is the second type of biometric information. For instance, the second type of biometric information may be heart rate information, blood oxygen status information, etc.

Taking the intelligent wearable device to be an intelligent ring as an example, as illustrated in FIG. 1, the wearable device body 11 is a ring body; the light source 12, the first photoelectric detection module 13 and the second photoelectric detection module 14 are disposed on an inner side of the ring body; the light source 12 is arranged towards the first photoelectric detection module 13; and the second photoelectric detection module 14 is disposed on a reflection path of light that is reflected by a finger.

A specific type of the light source 12 on the intelligent wearable device is not limited, as long as the light emitted by the light source can run through the human-body wearing part 100 and be reflected by the human-body wearing part 100. For instance, the light source 12 may adopt an LED source having the advantages of energy saving, high efficiency, no strobe and long service life. For instance, a near infrared light monochromatic LED source may be adopted, and near infrared light emitted by the light source has the wavelength of more than 0.4 μm and basically has no harm to the human body.

In the technical proposal as shown in FIG. 1 provided by the embodiments of the present disclosure, the light source 12 is arranged towards the first photoelectric detection module 13; the first photoelectric detection module 13 may detect transmitted light that passes through the finger; the second photoelectric detection module 14 is disposed on a reflection path of light that is reflected by the finger, and may detect the reflected light that is reflected by the finger; and the processing module 15 is configured to determine venous information of the finger according to the transmitted light detected by the first photoelectric detection module 13 and determine heart rate information and/or blood oxygen status information of the human body according to the reflected light detected by the second photoelectric detection module 14.

For instance, the principle for determining the venous information of the finger includes that: as hemachrome in blood has characteristic of absorbing near infrared light, the first photoelectric detection module may convert the received transmitted light into electric signals, and subsequently, a venous image of the finger is formed after data processing. The processing module may determine the venous information of the finger by adoption of processing means such as filtering, image binaryzation and thinning.

For instance, the principle for determining the heart rate information and the blood oxygen status information of the human body includes that: the second photoelectric detection module may convert the received reflected light into electric signals; when the heart rate of the human body changes, the frequency of the reflected light reflected by blood in the finger may also change correspondingly; and the processing module may determine the heart rate information of the human body by performing filtering, amplification and relevant algorithm routine processing on the electric signals. Similarly, when the blood oxygen status of the human body changes, the amount of the reflected light reflected by the blood in the finger may also change correspondingly, and the blood oxygen status information of the human body may be determined after processing the electric signals.

As illustrated in FIG. 2, in one embodiment of the present disclosure, the processing module 15 includes a pattern recognition chip 151. The pattern recognition chip 151 is configured to: calculate the venous information; compare the venous information with pre-stored venous information of the user; determine that an identity authentication of the user succeeds when the venous information is matched with the pre-stored venous information; and determine that the identity authentication of the user does not succeed when the venous information is not matched with the pre-stored venous information.

By adoption of the solution, the intelligent wearable device may be used for user identification, for instance, used for payment identification, login identification and the like, and has the advantages of high anti-falsification, high accuracy, high convenience, etc. The specific application scenario of identification is not limited. For instance, identification may be applied in the financial system, the cardless member management system, the data encryption system, the security system, the insurance system, the examination system, the medical system, the military aviation system, etc.

As illustrated in FIG. 2, in the embodiments of the present disclosure, the processing module 15 further includes a payment chip 152. The payment chip 152 is configured to execute a payment operation when the identity authentication of the user succeeds. The proposed solution can improve the payment operation safety of the intelligent wearable device.

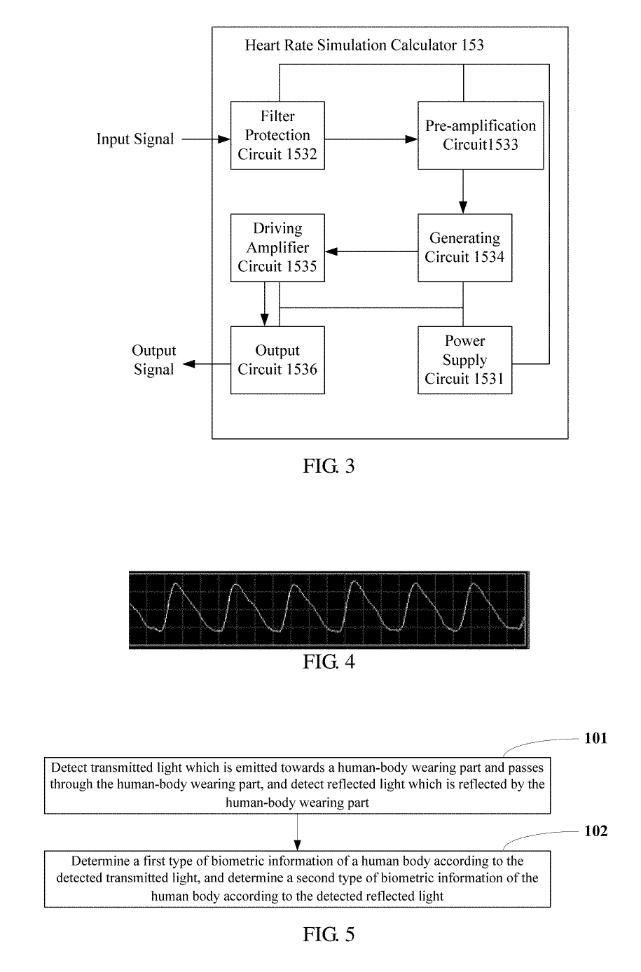

In the embodiments of the present disclosure, the processing module 15 further includes a heart rate simulation calculator 153 and is configured to determine the heart rate information and/or the blood oxygen status information of the human body according to the reflected light detected by the second photoelectric detection module, which includes: allowing the heart rate simulation calculator 153 to generate a heart rate simulation curve (as shown in FIG. 4) and/or heart rate values according to the reflected light detected by the second photoelectric detection module 14. The proposal allows the user to more directly read heart rate detection results.

A specific structural form of the heart rate simulation calculator 153 is not limited. As illustrated in FIG. 3, in the embodiment, the heart rate simulation calculator 153 includes a power supply circuit 1531, and a filter protection circuit 1532, a pre-amplification circuit 1533, a generating circuit 1534, a driving amplifier circuit 1535 and an output circuit 1536 which are respectively connected with the power supply circuit 1531 and sequentially connected with one another. The power supply circuit 1531 is configured to supply power for various components of the heart rate simulation calculator 153. The filter protection circuit 1532 is configured to perform filtering processing on input signals. For instance, the filter protection circuit 1532 is a filter. The pre-amplification circuit 1533 is configured to perform amplification processing on filtered signals. For instance, the pre-amplification circuit 1533 may be an amplifier. The generating circuit 1534 is configured to determine the second type of biometric information according to the reflected light. For instance, the generating circuit 1534 may determine and generate parameters such as the heart rate simulation curve and/or the heart rate values. The driving amplifier circuit 1535 may perform amplification processing on the second type of biometric information generated by the generating circuit 1534, and subsequently, the output circuit 1536 is configured to output the second type of biometric information.

Continuing to refer to FIG. 2, the processing module 15 may further include a wireless interface 154, which is configured to send the first type of biometric information and the second type of biometric information to a cloud device or a terminal device (e.g., a mobile phone). Thus, personal health information can be managed and monitored through the cloud device or the terminal device, and the cloud device or the terminal device may further feed back relevant suggestions or comments to the processing module, so that the intelligent wearable device can remind users to pay attention to their own health status. Of course, the processing module 15 may also receive data from the first photoelectric detection module 13 and the second photoelectric detection module through the wireless interface 154.

The intelligent wearable device provided by the embodiments of the present disclosure may further comprise one or more processors and more or more memories. The processor may process data signals and include various computing structures, e.g., a complex instruction set computer (CISC) structure, a reduced instruction set computing (RISC) structure or a structure for implementing multiple instruction set combinations. The memory may store instructions and/or data executed by the processor. The instructions and/or data may include codes and are configured to achieve some functions or all the functions described in the embodiments of the present disclosure. For instance, the memory includes a dynamic random access memory (DRAM), a static random access memory (SRAM), a flash memory, an optical memory or other memories known by those skilled in the art. Of course, the processor and the memory may also be included in the processing module 15.

As illustrated in FIG. 5, the embodiments of the present disclosure further provide a detection method of biometric information used in the intelligent wearable device. The method comprises the following steps:

Step S101: detecting transmitted light which is emitted towards a human-body wearing part and passes through the human-body wearing part, and detecting reflected light which is reflected by the human-body wearing part.

Step S102: determining a first type of biometric information of the human body according to the detected transmitted light, and determining a second type of biometric information of the human body according to the detected reflected light.

In the above solution, a same light source is adopted to provide detection light for the first photoelectric detection module and the second photoelectric detection module, so that multiple types of biometric information can be simultaneously detected, and hence the efficiency of the intelligent wearable device in detecting the biometric information can be effectively improved.

For instance, the first type of biometric information may include venous information, and the second type of biometric information may include heart rate information and/or blood oxygen status information.

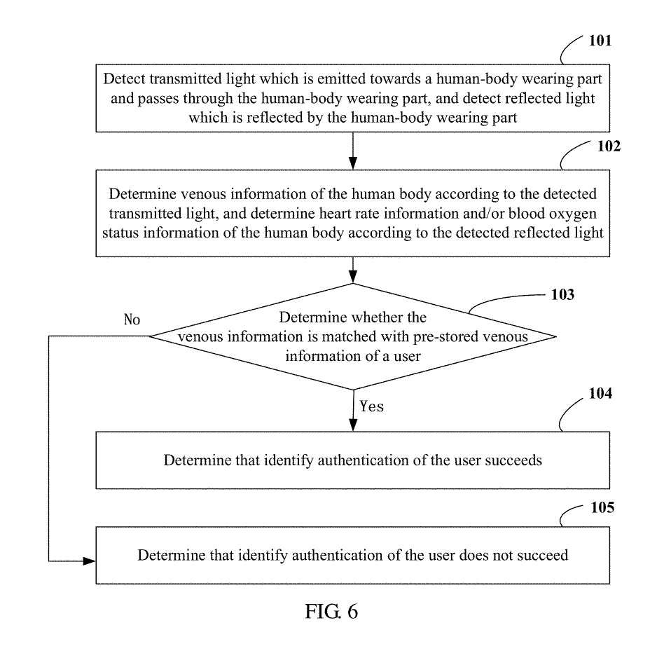

As illustrated in FIG. 6, after the steps S101 and S102, the detection method may further comprise the following steps:

Step S103: determining whether the venous information is matched with pre-stored venous information of the user; executing step S104 if the venous information is matched with the pre-stored venous information of the user; otherwise, executing step S105 if the venous information is not matched with the pre-stored venous information of the user.

Step S104: determining that the identity authentication of the user succeeds.

Step S105: determining that the identity authentication of the user does not succeed.

The intelligent wearable device may be used for user identification and has high safety and convenience.

For instance, after the step S104, the detection method may further comprise: performing a payment operation when the identity authentication of the user succeeds. The solution can improve the payment operation safety of the intelligent wearable device.

For instance, the heart rate information includes a heart rate simulation curve and/or heart rate values.

In the present disclosure, terms such as “first”, “second” and the like used in the present disclosure do not indicate any sequence, quantity or significance but only for distinguishing different constituent parts. Also, the terms such as “a,” “an,” or “the” etc., are not intended to limit the amount, but indicate the existence of at lease one. The terms “comprises,” “comprising,” “includes,” “including,” etc., are intended to specify that the elements or the objects stated before these terms encompass the elements or the objects and equivalents thereof listed after these terms, but do not preclude the other elements or objects.

Obviously, those skilled in the art may modify the disclosure in various ways without breaking away from the spirits and scope of the disclosure. And so, if these changes and variations of the disclosure also fall within the scope of the claims or their equivalent technologies, the disclosure intends to include these changes and variations.

The foregoing are merely specific embodiments of the disclosure, but not limitative to the protection scope of the disclosure. One skilled in the art could devise variations or replacements that within the scope and the spirit of the present disclosure, those variations or replacements shall belong to the protection scope of the disclosure. Thus, the protection scope of the disclosure shall be defined by the accompanying claims.

The present disclosure claims the benefits of Chinese patent application No. 201510822638.8, which was filed with the SIPO on Nov. 24, 2015 and is incorporated herein in its entirety by reference as part of this application.

1. An intelligent wearable device, comprising:
a wearable device body; and
a light source, a first photoelectric detection module, a second photoelectric detection module and a processing module disposed on the wearable device body, wherein:
the light source is configured to emit light towards a human-body wearing part; the light includes transmitted light that passes through the human-body wearing part and reflected light reflected by the human-body wearing part;
the first photoelectric detection module is configured to detect the transmitted light;
the second photoelectric detection module is configured to detect the reflected light; and
the processing module is connected with the first photoelectric detection module and the second photoelectric detection module, and the processing module is configured to determine a first type of biometric information of a human body according to the transmitted light detected by the first photoelectric detection module and determine a second type of biometric information of the human body according to the reflected light detected by the second photoelectric detection module.

2. The intelligent wearable device according to claim 1, wherein the first type of biometric information includes venous information, and the second type of biometric information includes at least one of heart rate information or blood oxygen status information.

3. The intelligent wearable device according to claim 2, wherein:
the processing module includes a pattern recognition chip; and
the pattern recognition chip is configured to: calculate the venous information; and determine whether the venous information is matched with pre-stored venous information of a user by comparing the venous information with the pre-stored venous information.

4. The intelligent wearable device according to claim 3, wherein the pattern recognition chip is also configured to:
determine that identity authentication of the user succeeds when the venous information is matched with the pre-stored venous information; or
determine that the identity authentication of the user does not succeed when the venous information is not matched with the pre-stored venous information.

5. The intelligent wearable device according to claim 4, wherein the processing module further includes a payment chip which is configured to perform a payment operation when the identity authentication of the user succeeds.

6. The intelligent wearable device according to claim 2, wherein:
the processing module includes a heart rate simulation calculator; and
the processing module is configured to determine the heart rate information and/or the blood oxygen status information of the human body according to the reflected light detected by the second photoelectric detection module, which includes: the heart rate simulation calculator generates a heart rate simulation curve and/or heart rate values according to the reflected light detected by the second photoelectric detection module.

7. The intelligent wearable device according to claim 6, wherein the heart rate simulation calculator includes a power supply circuit and a filter protection circuit, a pre-amplification circuit, a generating circuit, a driving amplifier circuit and an output circuit which are respectively connected with the power supply circuit and sequentially connected with one another.

8. The intelligent wearable device according to claim 1, wherein the processing module includes a wireless interface and is configured to send the first type of biometric information and the second type of biometric information to at least one of a cloud device or a terminal device.

9. The intelligent wearable device according to claim 1, wherein the light source is a light-emitting diode (LED) source.

10. The intelligent wearable device according to claim 9, wherein the LED source is a near infrared light monochromatic LED source.

11. The intelligent wearable device according to claim 1, wherein the intelligent wearable device is an intelligent ring.

12. The intelligent wearable device according to 10, wherein the wearable device body is a ring body; the light source, the first photoelectric detection module and the second photoelectric detection module are disposed on an inner side of the ring body; the light source is arranged towards the first photoelectric detection module; and the second photoelectric detection module is disposed on a reflection path of light reflected by the human-body wearing part.

13. A detection method of biometric information for an intelligent wearable device, comprising:
detecting transmitted light which is emitted towards a human-body wearing part and passes through the human-body wearing part, and detecting reflected light which is reflected by the human-body wearing part;
determining a first type of biometric information according to the detected transmitted light; and
determining a second type of biometric information according to the detected reflected light.

14. The method according to claim 13, wherein the first type of biometric information includes venous information, and the second type of biometric information includes at least one of heart rate information or blood oxygen status information.

15. The method according to claim 14, further comprising:
calculating the venous information; and
determining whether the venous information is matched with pre-stored venous information of a user by comparing the venous information with the pre-stored venous information.

16. The method according to claim 15, further comprising:
determining that identity authentication of the user succeeds when the venous information is matched with the pre-stored venous information; or
determining that the identity authentication of the user does not succeed when the venous information is not matched with the pre-stored venous information.

17. The method according to claim 16, further comprising:
performing a payment operation when the identity authentication of the user succeeds.

18. The method according to claim 14, wherein the heart rate information includes at least one of a heart rate simulation curve or heart rate values.

19. The intelligent wearable device according to claim 2, wherein the intelligent wearable device is an intelligent ring.

20. The intelligent wearable device according to claim 3, wherein the intelligent wearable device is an intelligent ring.
